SEQUENCE LISTING (3 sequences)

SEQ ID NO: 1 - protein, 36 aa, unknown
  TAT-C3P
  TAT-C3P (1)..(36)
GRKKRRQRRRPQEESNKEAREDAELDAEIELEMAQG

SEQ ID NO: 2 - protein, 36 aa, unknown
  unkown
  TAT-C3D (1)..(36)
GRKKRRQRRRPQAVSSPARSGEPLHALSPRGTARSP

SEQ ID NO: 3 - protein, 42 aa, unknown
  His-tagged TAT-C3P
  His-tagged TAT-C3P (1)..(42)
GRKKRRQRRRPQEESNKEAREDAELDAEIELEMAQGHHHHHH

(19) United States
(12) Patent Application Publication
(10) Pub. No.: US 2022/0133847 A1
(43) Pub. Date: May 5, 2022

(54) COMPOSITIONS AND METHODS FOR ALLEVIATING PAIN

(71) Applicant: UNM RAINFOREST INNOVATIONS, Albuquerque, NM (US)

(72) Inventors: Sascha Alles, Albuquerque, NM (US); Karin Westlund High, Albuquerque, NM (US)

(21) Appl. No.: 17/517,045

(22) Filed: Nov. 2, 2021

Related U.S. Application Data
(60) Provisional application No. 63/109,206, filed on Nov. 3, 2020.

(51) Int. Cl. A61K 38/17; C12N 15/113; C07K 16/28; A61P 29/00

(52) U.S. Cl. CPC A61K 38/17; C12N 15/1138; C12N 2320/30; A61P 29/00; C12N 2310/11; C07K 16/28

(57) ABSTRACT

A therapeutic composition includes a Caᵥ3.3 inhibitor and a pharmaceutically acceptable carrier. In some embodiments the Caᵥ3.3 inhibitor can include a polypeptide such as, for example, the amino acid sequence of SEQ ID NO:1, an antibody, or fragment thereof. In some embodiments, the Caᵥ3.3 inhibitor can include an inhibitory polynucleotide such as, for example, an anti-sense oligonucleotide that either hybridizes to a portion of Cacnali or hybridizes to an RNA transcribed from a portion of Cacnali. The therapeutic composition can be administered to a subject to alleviate pain in the subject or to increase the subject's mechanical withdrawal threshold. The composition can be used to reduce Caᵥ3.3 expression in a cell.

### CROSS-REFERENCE TO RELATED APPLICATION

This application claims the benefit of U.S. Provisional Patent Application No. 63/109,206, filed on Nov. 3, 2020, of which is incorporated herein by reference in its entirety.

### GOVERNMENT FUNDING

This invention was made with government support under DE028096 awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “0310-000163US01_ST25.txt” having a size of 2 KB and created on Oct. 18, 2021. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, a method for treating pain in a subject. Generally, the method includes administering a Caᵥ3.3 inhibitor to a subject in an effective amount to alleviate pain.

In some embodiments, the pain is chronic pain. In some embodiments, the chronic pain is trigeminal neuropathic pain.

In some embodiments, the Caᵥ3.3 inhibitor is more effective for female subject than a male subject.

In another aspect, this disclosure describes a method for reducing Caᵥ3.3 expression in a cell. Generally, the method includes contacting a cell with a Caᵥ3.3 inhibitor in an amount affective to reduce Caᵥ3.3 expression.

In some embodiments, reducing the expression of Caᵥ3.3 includes reducing Caᵥ3.3 in the lysate of whole trigeminal ganglion cells.

In some embodiments, the Caᵥ3.3 inhibitor is more effective at reducing Caᵥ3.3 expression in a cell isolated from a female than in a cell isolated from a male.

In another aspect, this disclosure describes a method for increasing the mechanical withdrawal threshold in a subject. Generally, the method includes administering a Caᵥ3.3 inhibitor to a subject in an amount effective to increase the mechanical withdrawal threshold of the subject.

In some embodiments, the Caᵥ3.3 inhibitor is more effective at increasing the mechanical withdrawal of a female subject than a male subject.

In any of the previously describe aspects and embodiments, the Caᵥ3.3 inhibitor may include a polypeptide. In some embodiments, the polypeptide includes the amino acid sequence of SEQ ID NO:1 or a structurally similar amino acid sequence. In some embodiments, the peptide includes an antibody or a binding fragment of an antibody.

In any of the previously describe aspects and embodiments, the Caᵥ3.3 inhibitor may include an inhibitory polynucleotide. In some embodiments, the polynucleotide includes an anti-sense oligonucleotide that hybridizes to at least a portion of Cacnali or an RNA transcribed from a portion of Cacnali.

In another aspect, this disclosure describes a Caᵥ3.3 inhibitor composition useful for alleviating pain. Generally, the composition includes a Caᵥ3.3 inhibitor in the form of an inhibitory polynucleotide and a pharmaceutically acceptable carrier.

In some embodiments, the inhibitory polypeptide includes an anti-sense oligonucleotide that hybridizes to at least a portion of Cacnali or an RNA transcribed from a portion of Cacnali.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Effect of a Caᵥ3.3 inhibiting peptide on mechanical allodynia in FRICT-ION mice. (A) The Caᵥ3.3-inhibiting peptide TAT-C3P (SEQ ID NO:1) was administered to male mice via intraperitoneal injection at a dose of 10 mg/kg and the mice were monitored every hour for up to five hours post-injection. For control experiments, TAT-C3D (SEQ ID NO:2), a peptide that does not inhibit Caᵥ3.3 channels was used. (B) The Caᵥ3.3-inhibiting peptide TAT-C3P (SEQ ID NO:1) was administered to female mice via intraperitoneal injection at a dose of 10 mg/kg and monitored every hour for up to five hours post-injection. For control experiments, TAT-C3D (SEQ ID NO:2), a peptide that does not block Caᵥ3.3 channels was used. * p<0.05 compared to FRIC+TAT-C3D, **** p<0.0001 compared to FRIC+TAT-C3D

FIG. 2. Effect of a Caᵥ3.3-inhibiting peptide (SEQ ID NO:1) on mechanical allodynia in FRICT-ION mice. Comparison of effectiveness of 10 mg/kg Caᵥ3.3-inhibiting peptide in male and female mice. **** p<0.0001 compared to FRIC+TAT-C3D, ++p<0.01 compared to males with FRIC+TAT-C3D.

FIG. 3. Protein levels of Caᵥ3.3 in male and female FRICT-ION mice following a Caᵥ3.3-inhibiting peptide injection. (A) Caᵥ3.3 versus actin levels in whole trigeminal ganglia (TG) lysates of naïve, male FRICT-ION mice injected with the control TAT-C3D peptide (SEQ ID NO:2), female FRICT-ION mice injected with the control TAT-C3D peptide (SEQ ID NO:2), male FRICT-ION mice injected with Caᵥ3.3-inhibiting TAT-C3P peptide (SEQ ID NO:1), and female FRICT-ION mice injected with Caᵥ3.3-inhibiting TAT-C3P peptide (SEQ ID NO:1) (n=3 mice per group). (B) Normalized ratio of intensity of TAT-C3P-injected to the control TAT-C3D-injected male and female FRICT-ION mice. (*p<0.05, t-test).

FIG. 4. Direct effect of the TAT-C3P (SEQ ID NO:1) on TG neurons from FRICT-ION mice. (A) Resting membrane potential was hyperpolarized under treated (n=25) compared to control (n=21) conditions in females only. Current clamp traces are shown for stepwise current injections from −100 to +190 pA. (B) Immunohistochemistry staining of female TG primary cell culture. Confirmation that the Cav3.3-inhibiting peptide reduces expression of Cav3.3 (large spots). DAPI staining (flecks) is used to visualize cell nuclei. Scale bar (top left)=200 μm.

FIG. 5. The TAT-C3P peptide (SEQ ID NO:1) penetrates the central nervous system following intraperitoneal injection in FRICT-ION mice. (A) Bands were detected for His-tagged TAT-C3P in TG and medullary tissues obtained from His-tagged TAT-C3P injected male FRICT-ION mice, but not for controls injected with the TAT-C3P peptide (SEQ ID NO:1). Animals were sacrificed, and tissues were harvested four hours post-injection. (B) FRICT-ION mice experience the same level of anti-allodynic effect with His-tagged TAT-C3P as non-His-tagged TAT-C3P (n=2 mice, ****p<0.0001, 2-way ANOVA).

### DETAILED DESCRIPTION OF ILLUSTRATIVE EMBODIMENTS

This disclosure describes compositions and methods for alleviating pain, especially chronic pain. Chronic pain is experienced by one in five people globally. Opioids, which are very effective for acute pain, are not very effective for chronic pain. Therefore, the problem of chronic pain is compounded by opioid abuse.

A particularly traumatic chronic pain type is trigeminal neuropathic pain, which is caused by inflammation and demyelination of the trigeminal nerve pathways. This disclosure describes compositions and methods for treating trigeminal neuropathic pain. Generally, the compositions include a compound that inhibits the Caᵥ3.3 T-type calcium channel. Further, this disclosure describes a potential sex difference in the role of Caᵥ3.3 in trigeminal neuropathic pain, which may define a subpopulation for whom the compositions may be particularly effective.

In this disclosure, differential gene expression pattern in the trigeminal ganglia (TG) of mice experiencing trigeminal neuropathic pain compared to naïve mice were analyzed using the FRICT-ION mouse model. The FRICT-ION (Foramen Rotundum Inflammatory Constriction Trigeminal InfraOrbital Nerve) mouse model is a highly robust, long-lasting, and easily induced animal model of trigeminal neuropathic pain that accurately depicts the kind of pain suffered by patients (Montera, M. A. and Westlund, K. N. (2020). Bio-protocol 10(8): e3591). The FRICT-ION model persists through long time frames suitable for testing therapeutics for chronic pain (e.g., greater than six months). Differential gene expression analysis identified the Caᵥ3.3 T-type calcium channel as being upregulated in the trigeminal ganglia of mice in the FRICT-ION mouse model. Behavioral pharmacology experiments using a selective Caᵥ3.3-inhibiting peptide show that mechanical allodynia is alleviated in both male and female FRICT-ION mice. Furthermore, the Caᵥ3.3-inhibiting peptide may be more effective in female mice than male mice in alleviating mechanical allodynia in the FRICT-ION model.

Expression of the Cacnali gene, encoding for the Caᵥ3.3 ion channel, is upregulated in the trigeminal ganglia of FRICT-ION mice. RNAseq was used to analyze gene expression in the trigeminal ganglia (TG) to determine changes occurring at the transcriptional level in FRICT-ION mice compared to control mice. Expression of the Cacnali gene increased by 40% compared to the control (p<0.05, paired t-test; Table 1). Cacnali was one of only a few ion channel genes upregulated while most were downregulated at ten weeks (Table 1).

TABLE 1
RNAseq analysis showing up- and downregulated ion channel
gene expression in the TG isolated from FRICT-ION mice

| | | | |
|---|---|---|---|
| Gene | | | |
| symbol | Name | % Change | p-value |

Upregulated ion channel genes

| | | | |
|---|---|---|---|
| Cacna1g | calcium channel; voltage-dependent; T type; alpha 1G | 35.6% | 0.358 |
| | subunit | | |
| Cacna1h | calcium channel; voltage-dependent; T type; alpha 1H | 10.9% | 0.160 |
| | subunit | | |
| Cacna1i | calcium channel; voltage-dependent; alpha 1I subunit | 39.8% | 0.0276 |
| Gapdh | glyceraldehyde-3-phosphate dehydrogenase | 3.47% | 0.590 |
| Kcnh6 | potassium voltage-gated channel; subfamily H (eag- | 20.3% | 0.0449 |
| | related); member 6 | | |
| Clic4 | chloride intracellular channel 4 (mitochondrial) | 25.1% | 0.0208 |
| Kcnj10 | potassium inwardly-rectifying channel; subfamily J; | 37.0% | 0.000219 |
| | member 10 | | |
| Hvcn1 | hydrogen voltage-gated channel 1 | 60.4% | 0.0370 |
| Kcnj16 | potassium inwardly-rectifying channel; subfamily J; | 81.5% | 0.0102 |
| | member 16 | | |

Downregulated ion channel genes

| | | | |
|---|---|---|---|
| Kcnb2 | potassium voltage gated channel; Shab-related | −44.2% | 0.0270 |
| | subfamily; member 2 | | |
| Kcnv1 | potassium channel; subfamily V; member 1 | −40.0% | 0.0112 |
| Kcnh5 | potassium voltage-gated channel; subfamily H (eag- | −35.3% | 0.0194 |
| | related); member 5 | | |
| Kcnh7 | potassium voltage-gated channel; subfamily H (eag- | −34.4% | 0.0302 |
| | related); member 7 | | |
| Kcnmb2 | potassium large conductance calcium-activated | −32.0% | 0.0468 |
| | channel; subfamily M; beta member 2 | | |
| Scn1a | sodium channel; voltage-gated; type I; alpha | −25.2% | 0.00392 |
| Kcnq3 | potassium voltage-gated channel; subfamily Q; | −21.2% | 0.0321 |
| | member 3 | | |
| Kcna2 | potassium voltage-gated channel; shaker-related | −18.3% | 0.0341 |
| | subfamily; member 2 | | |
| Scn9a | sodium channel; voltage-gated; type IX; alpha | −16.1% | 0.0414 |
| Nalcn | sodium leak channel; non-selective | −14.4% | 0.0172 |
| Trpm7 | transient receptor potential cation channel; subfamily | −13.6% | 0.0171 |
| | M; member 7 | | |

The expression of all Cav3 subtypes, Cacnalg, Cacnalh, Cacnali are indicated. Cacnali expression increase is statistically significantly (p<0.05, paired t-test), whereas that of Cacnalg and Cacnalh are not altered (p>0.05, paired t-test) compared to naïve controls. Expression of housekeeping gene Gapdh is not significantly changed compared to controls (p>0.05, paired t-test). TG were isolated at ten weeks post-injury. This analysis is based on RNA obtained from whole TG tissue of 3 FRICT-ION and three naïve male mice. Percentage changes with corresponding p-values are indicated to significant figures.

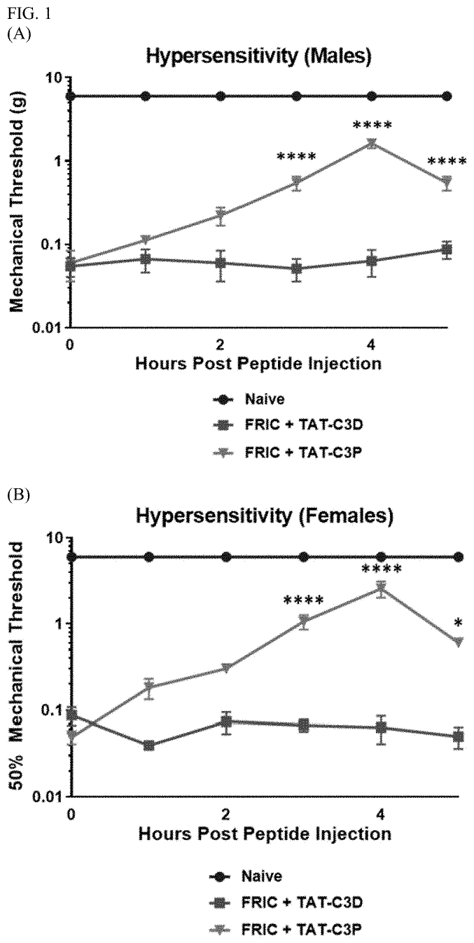

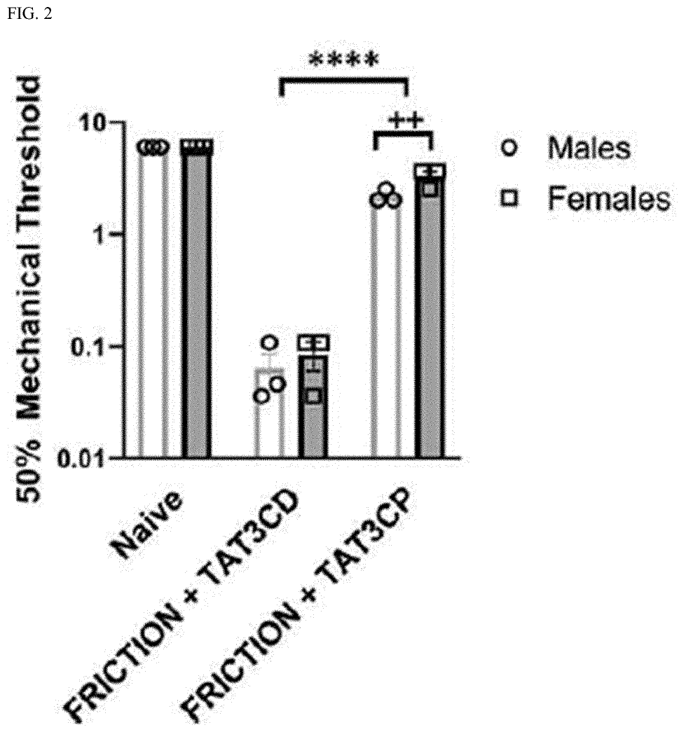

Inhibiting the Caᵥ3.3 ion channel is a strategy for alleviating neuropathic pain. A peptide that selectively inhibits Caᵥ3.3 alleviates mechanical allodynia in FRICT-ION mice. The Caᵥ3.3-inhibiting peptide TAT-C3P (SEQ ID NO:1; Cmarko L and Weiss N, 2020. Mol Brain 13:95) was used in the FRICT-ION model and demonstrated a significant increase (p<0.0001, 2-way ANOVA with posthoc Tukey's test) in mechanical withdrawal thresholds in both male (FIG. 1A, FIG. 2) and female (FIG. 1B, FIG. 2) mice compared to the control peptide TAT-C3D (SEQ ID NO:2), which does not inhibit Caᵥ3.3. Further, inhibiting Caᵥ3.3 resulted in a more significant increase in withdrawal threshold in female than male mice, indicating reduction of pain-related behavior (FIG. 2). Comparing data at the four-hour time point post-injection, female FRICT-ION mice withdrawal thresholds were significantly higher than male FRICT-ION mice withdrawal thresholds, indicating less pain-related behavior (p<0.01, 2-way ANOVA with posthoc Tukey's test).

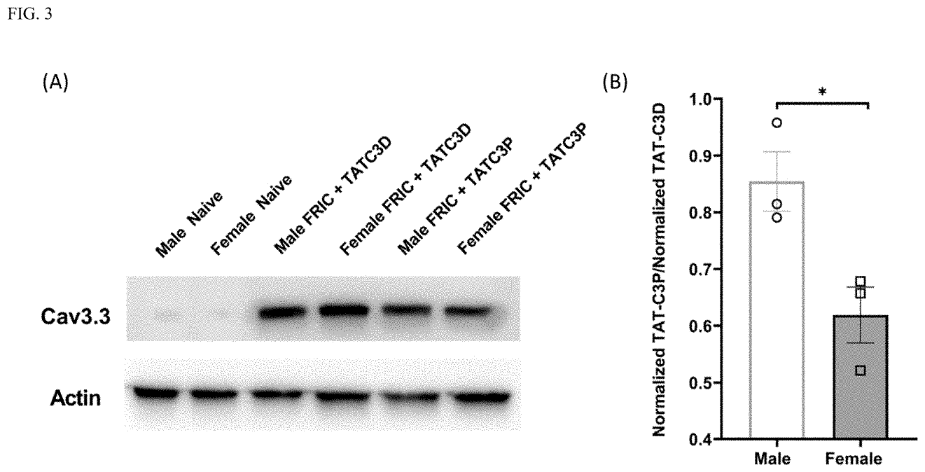

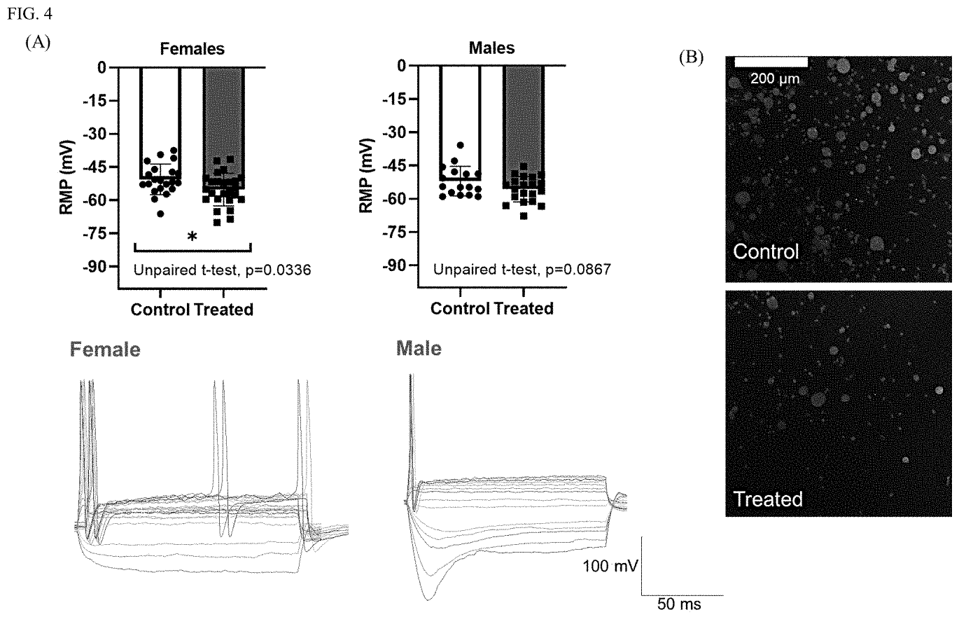

Treatment of FRICT-ION mice with the Caᵥ3.3 inhibitor TAT-C3P (SEQ ID NO: 1), decreased the Caᵥ3.3 protein levels in the trigeminal ganglia (TG). Western blot analysis of Caᵥ3.3 protein levels from whole TG lysates was performed (n=3 mice per group; FIG. 3A) to compare the Caᵥ3.3 protein levels in naïve mice, FRICT-ION mice treated with control (TAT-C3D, SEQ ID NO:2), and FRICT-ION mice treated with a Caᵥ3.3 inhibitor (TAT-C3P, SEQ ID NO:1). Signal intensity for Caᵥ3.3 was near absent in naïve mice and was strongly present in FRICT-ION mice treated with the TAT-C3D (SEQ ID NO: 2) control peptide. Thus, inhibiting Caᵥ3.3 reduces the nerve-injury-related increase. The differential effect in males and females was compared by analyzing the fold change, normalized to actin, of Caᵥ3.3 signal intensity for TAT-C3P-injected mice to TAT-C3D-injected mice between male and female FRICT-ION mice (FIG. 4A). A significant difference (p<0.05, t-test) between these fold-changes with female mice displaying a smaller fold-change than male mice, indicates that TAT-C3P (SEQ ID NO:1) is more effective at reducing Caᵥ3.3 expression in female mice than in male mice. The reduced Caᵥ3.3 upregulation in females parallels the improved pain-related behavior in the females compared to males.

TAT-C3P (SEQ ID NO:1) acts directly on trigeminal ganglia neurons from FRICT-ION mice. TAT-C3P (SEQ ID NO:1) acts directly on neurons following a one-hour treatment in vitro, which correlates with its anti-allodynic actions in vivo. TAT-C3P is effective in mitigating neuronal excitability through a hyperpolarizing effect on resting membrane potential of TG neurons. However, this effect appears to be exclusively on neurons from female FRICT-ION mice (FIG. 4A). The hyperpolarizing effect of TAT-C3P on neurons may mitigate neuronal hyperexcitability underlying chronic pain in a physiological setting. These effects provide a mechanism of action of TAT-C3P on neurons from female FRICT-ION mice. In chronic pain states, the immune cell populations of male and female mice, such as T-cells and microglia, have differential roles. Thus, the mechanism of action of TAT-C3P may be centrally rather than peripherally mediated in male nerve-injured mice. Immunohistochemistry data indicates reduced expression of Cav3.3 following TAT-C3P treatment (FIG. 4B), which agrees with Western blot data.

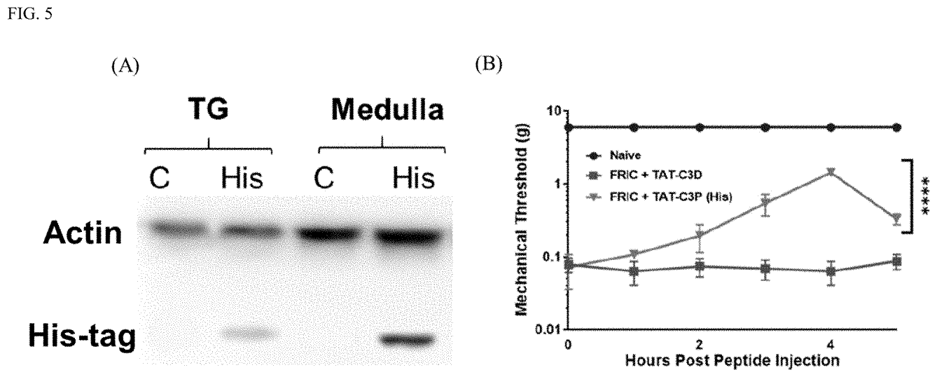

TAT-C3P penetrates the central nervous system (CNS). A His-tagged version of TAT-C3P showing that the peptide penetrates the CNS following intraperitoneal injection (FIGS. 5A and 5B).

Thus, this disclosure identifies Cacnali, encoding the Caᵥ3.3 channel, as an ion-channel-encoding gene that is upregulated ten weeks post-trigeminal nerve injury in the whole TG compared to controls. Many other ion channel mRNAs were downregulated at this chronic ten-week time point. Further, Caᵥ3.3 protein levels are elevated in the trigeminal ganglia of mice in the FRICT-ION model. Selectively inhibiting Caᵥ3.3 is, therefore, an effective strategy for alleviating neuropathic pain in both male and female mice. Additionally, inhibiting Caᵥ3.3 appears to be more effective in alleviating mechanical allodynia in female mice than in male mice.

Caᵥ3.3 belongs to the family of T-type Ca²⁺ channels, which are expressed in small-diameter and medium-diameter primary afferent neurons. T-type Ca²⁺ channels regulate neuronal excitability and are involved in neuropathic pain. Inhibiting or attenuating all the T-type Ca²⁺ channels is an effective strategy for mitigating behavioral signs of neuropathic pain in animal models. However, the pan-T-type Ca²⁺ channel blocker ethosuximide has been tested in clinical trials for neuropathic pain, but the trial was halted due to adverse events experienced by patients.

A selective inhibitor of Caᵥ3.3 channels, the TAT-C3P peptide (SEQ ID NO:1), can reduce protein levels of Caᵥ3.3 in the trigeminal ganglia of FRICT-ION mice injected with TAT-C3P (SEQ ID NO: 1). This result suggests that TAT-C3P may help normalize Caᵥ3.3 to baseline levels in FIRCT-ION mice with nerve injuries. In addition, female mice injected with TAT-C3P (SEQ ID NO:1) displayed a greater decrease of Caᵥ3.3 protein levels compared to control TAT-C3D (SEQ ID NO: 2) peptide than male mice. This result suggests that the TAT-C3P peptide is more effective in reducing Caᵥ3.3 levels in female mice, which may explain the behavioral sex differences of the TAT-C3P peptide (SEQ ID NO:1).

Thus, this disclosure describes a Caᵥ3.3 as a target for alleviating trigeminal neuropathic pain, pharmaceutical compositions that include an inhibitor of Caᵥ3.3, and methods of treating pain (e.g., trigeminal neuropathic pain and other chronic pain symptoms).

In some embodiments, the Caᵥ3.3 inhibitor can be a protein or peptide (e.g., SEQ ID NO:1, antibody, affibody etc.) or fragment thereof. As used herein, the term “antibody” refers to a molecule that contains at least one antigen binding site that immunospecifically binds to a particular antigen target of interest—e.g., Caᵥ3.3. The term “antibody” thus includes, but is not limited to, a full length antibody and/or its variants, a fragment thereof, a peptibody and/or variants thereof, a monoclonal antibody (including a full-length monoclonal antibody), a multispecific antibody (e.g., a bispecific antibody) formed from at least two intact antibodies, a human antibody, a humanized antibody, and/or an antibody mimetic that mimic the structure and/or function of an antibody or a specified fragment or portion thereof, including a single chain antibody and/or fragments thereof. Thus, as used herein, the term “antibody” encompasses antibody fragments capable of binding to a biological molecule (such as an antigen or receptor) or a portion thereof, including, but not limited to, an Fab, an Fab′ an F(ab′)2, a pFc′, an Fd, a single domain antibody (sdAb), a variable fragment (Fv), a single-chain variable fragment (scFv), or a disulfide-linked Fv (sdFv); a diabody or a bivalent diabody; a linear antibody; a single-chain antibody molecule; or a multispecific antibody formed from antibody fragments. The antibody can be of any type (e.g., IgG, IgE, IgM, IgD, IgA and IgY), class (e.g., IgG1, IgG2, IgG3, IgG4, IgA1 and IgA2), or subclass.

In some embodiments, the Caᵥ3.3 inhibitor is a Caᵥ3.3 targeting peptide, such as, for example, the amino acids of SEQ ID NO: 1 or a structurally similar peptide.

As used herein, a polypeptide is “structurally similar” to a reference polypeptide if the amino acid sequence of the polypeptide possesses a specified amount of identity compared to the reference polypeptide. Structural similarity of two polypeptides can be determined by aligning the residues of the two polypeptides (for example, a candidate polypeptide and the polypeptide of, for example, SEQ ID NO: 1) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate polypeptide is the polypeptide being compared to the reference polypeptide (e.g., SEQ ID NO:1). A candidate polypeptide can be isolated, for example, from an animal, or can be produced using recombinant techniques, or chemically or enzymatically synthesized.

A pair-wise comparison analysis of amino acid sequences can be carried out using the BESTFIT algorithm in the GCG package (version 10.2, Madison Wis.). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a SEQ. ID NO: 1 polypeptide may be selected from other members of the class to which the amino acid belongs. For example, it is well-known in the art of protein biochemistry that an amino acid belonging to a grouping of amino acids having a particular size or characteristic (such as charge, hydrophobicity, and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH₂. Likewise, biologically active analogs of a polypeptide containing deletions or additions of one or more contiguous or noncontiguous amino acids that do not eliminate a functional activity of the polypeptide are also contemplated.

A Caᵥ3.3 targeting peptide as described herein can include a polypeptide with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to SEQ ID NO:1.

A Caᵥ3.3 targeting peptide as described herein also can be designed to provide additional sequences, such as, for example, the addition of added C-terminal or N-terminal amino acids that can, for example, facilitate purification by trapping on columns or use of antibodies. Such tags include, for example, histidine-rich tags that allow purification of polypeptides on nickel and cobalt columns. Such gene modification techniques and suitable additional sequences are known in the molecular biology arts. In some embodiments, the additional sequences may be cleaved from the peptide resulting in a scar or no scar.

In some embodiments, a Caᵥ3.3 targeting peptide as described herein also may include N-terminal or C-terminal functionalities other than a carboxylic acid or free amine. For example, the C-terminus, N-terminus, or both, of a Caᵥ3.3 targeting peptide may be acylated, for example, acetylated. A nanoparticle-encapsulated TAT-C3P peptide (e.g., encapsulation using fatty acids, PEGylation, crotonylation, etc.) may improve bioavailability of the peptide and, therefore, may prolong the anti-allodynic effectiveness of the TAT-C3P peptide. In addition, nanoparticle-encapsulation and targeting using endogenous ligands of certain receptors involved in pain pathways may allow one to target the TAT-C3P peptide only to specific neurons or cell types. This may improve the potential side effect profile of long-term administration of the TAT-C3P peptide even though no major side effects of TAT-C3P have been observed.

In another aspect, this disclosure describes polynucleotides that encode any of the polypeptides described herein, and the complements of such polynucleotide sequences. Given the amino acid sequence of the SEQ ID NO: 1 polypeptide described herein, a person of ordinary skill in the art can determine the full scope of polynucleotides that encode that amino acid sequence using conventional, routine methods.

In other embodiments, the Caᵥ3.3 inhibitor can be an inhibitory polynucleotide such as, for example, an anti-sense oligonucleotide inhibitor such as, for example, an anti-sense oligonucleotide previously described (Wen et al., 2006. Acta Pharmacol Sin. 27:1547-1552). Generally, an inhibitory polynucleotide is complementary to at least a portion of Cacnali, or an RNA transcribed from a portion of Cacnali so that the inhibitory polynucleotide hybridizes with Cacnali or an RNA transcribed from a portion of Cacnali and interferes with expression. The inhibitory polynucleotide may be DNA or RNA, single-stranded or double-stranded. Exemplary inhibitory polynucleotides include, but are not limited to, an mRNA, an inhibitory RNA (e.g., an antisense RNA, a microRNA (miRNA), a small interfering RNA (siRNA), a short hairpin RNA (shRNA), an agomiR, an antagomiR, a modified mRNA, a loop-engineered modified mRNA, or a combination thereof.

In some embodiments, the inhibitory polynucleotide sequences may be modified to include modifications on the 5′ end, 3′ end, or both ends. In some embodiments, modifications to the 5′ end, 3′ end, or both ends may increase the stability and half-life of the inhibitory polynucleotide in physiological conditions. In some embodiments, these modifications may improve cellular delivery and/or uptake. Conjugation chemistries known in the art may be used to append modifications to one or more ends of the inhibitory polynucleotide sequence. Exemplary modifications include, but are not limited to, conjugation to polymers, conjugation to lipid nanoparticles, conjugation to polymer nanoparticles, conjugation to cationic dendrimers, conjugation to lipids, conjugations to glycans, conjugation to peptides, conjugation to aptamers, conjugation to antibodies, conjugation to serum protein, and conjugation to small organic molecule.

In some embodiments, the sugar-phosphate backbone of the inhibitory polynucleotide may be modified. In some embodiments, the sugar-phosphate backbone modifications may increase the stability and half-life of the inhibitory polynucleotide in physiological conditions. Exemplary modifications include, but are not limited to, phosphorothioate backbone modifications, phosphorodiamidate morpholino oligomer backbone modifications, peptide nucleic acid modifications, alterations at the 2′ sugar position are the 2-O-Me and 2′-O-(2-methoxyethyl modifications, locked nucleic acids, constrained ethyl DNA modifications, and constrained tricyclo-DNA (tc-DNA). In some embodiments, sugar-phosphate backbone modification may be present at one or more of the nucleotides in the polynucleotide inhibitor sequence. A Caᵥ3.3 inhibitor (including, for example, SEQ ID NO:1) as the active agent, can be administered to a subject alone or in a pharmaceutical composition that includes the active agent and a pharmaceutically acceptable carrier. The active agent is administered to a subject such as a vertebrate, particularly a mammal, such as a human patient, companion animal, or domesticated animal, in an amount effective to produce the desired effect. Companion animals include, but are not limited to, dogs, cats, rabbits, and the like. Domesticated animals include, but are not limited to, pigs, chickens, horses, sheep, goats, cows, and the like. In some embodiments, the active agent is administered to a research subject. Examples of research subjects include, but not limited to, mice, rats, pigs, horses, rabbits, and monkeys.

A pharmaceutical composition may include one or more Caᵥ3.3 inhibitor. For example, a Caᵥ3.3 targeting peptide may be used in combination with a Caᵥ3.3 targeting antibody and/or a Caᵥ3.3 targeting oligonucleotide.

The pharmaceutical composition may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, an accessory agent, stabilizer, protein carrier, biological carrier compound and the like. Non-limiting examples of a protein carrier includes keyhole limpet hemocyanin (KLH), bovine serum albumin (BSA), ovalbumin, or the like. Non-limiting examples of a biological compound which may serve as a carrier include a glycosaminoglycan, a proteoglycan, and albumin. The carrier may be a synthetic compound, such as dimethyl sulfoxide or a synthetic polymer, such as a polyalkyleneglycol. Ovalbumin, human serum albumin, other proteins, polyethylene glycol, or the like may be employed as the carrier. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, e.g., the material may be administered to an individual along with a Caᵥ3.3 inhibitor without causing any undesirable biological effects or interacting in a deleterious manner with any of the other components of the pharmaceutical composition in which it is contained.

A Caᵥ3.3 inhibitor may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

A formulation may be conveniently presented in unit dosage form and may be prepared by methods well known in the art of pharmacy. Methods of preparing a composition with a pharmaceutically acceptable carrier include the step of bringing the Caᵥ3.3 inhibitor into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

Thus, a Caᵥ3.3 inhibitor may be provided in any suitable form including, but not limited to, a solution, a suspension, an emulsion, a spray, an aerosol, or any form of mixture. The composition may be delivered in formulation with any pharmaceutically acceptable excipient, carrier, or vehicle.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye.

Topical formulations include the active agent dissolved or suspended in one or more media such as mineral oil, petroleum, polyhydroxy alcohols, or other bases used for topical pharmaceutical formulations. For example, topical formulations may include a cream, an ointment, a paste, a lotion, a powder, a solid, an aerosolized foam, or a gel. Topical formulations may contain a permeation enhancer to increase the bioavailability of the active agent. Topical formulations may contain preservatives and/or emulsifiers. Topical formulations may be provided in the form of a transdermal patch or bandage, wherein the formulation is incorporated into a gauze or other structure and brought into contact with the skin. The formulation may further include one or more additives including such as, for example, an adjuvant, a skin penetration enhancer, a colorant, a fragrance, a flavoring, a moisturizer, a thickener, and the like.

Formulations of the present disclosure suitable for oral administration may be presented as discrete units such as tablets, troches, capsules, lozenges, wafers, or cachets, each containing a predetermined amount of the active agent as a powder or granules, as liposomes, or as a solution or suspension in an aqueous liquor or non-aqueous liquid such as a syrup, an elixir, an emulsion, or a draught. The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into preparations and devices in formulations that may or may not be designed for sustained release.

Formulations suitable for parenteral administration can include a sterile aqueous preparation of the active agent, or dispersions of sterile powders of the active agent, which are preferably isotonic with the blood of the recipient. Parenteral administration of a Caᵥ3.3 inhibitor (e.g., through an intravenous drip) is one form of administration. Isotonic agents that may be included in the liquid preparation include sugars, buffers, and sodium chloride. Solutions of the active agent may be prepared in water, optionally mixed with a nontoxic surfactant. Dispersions of the active agent may be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity may be achieved, for example, by using liposomes, by employing the appropriate particle size in the case of dispersions, or by using surfactants. Sterilization of a liquid preparation may be achieved by any convenient method that preserves the bioactivity of the active agent, preferably by filter sterilization. Preferred methods for preparing powders include vacuum drying and freeze drying of the sterile injectable solutions. Subsequent microbial contamination may be prevented using various antimicrobial agents, for example, antibacterial, antiviral, and antifungal agents including parabens, chlorobutanol, phenol, sorbic acid, thimerosal, and the like. Absorption of the active agent over a prolonged period may be achieved by including agents for delaying, for example, aluminum monostearate and gelatin.

In some embodiments, the pharmaceutical composition is administered to a subject such as a vertebrate, particularly a mammal, such as a human patient, companion animal, domesticated animal, or research animal to treat pain symptoms. In some embodiments, the pharmaceutical composition is administered to a subject to treat chronic pain. In some embodiments, the pharmaceutical composition is administered to treat trigeminal neuropathic pain. Trigeminal neuropathic pain is characterized by a constant aching or burning pain over part of the face. The pain may also be stabbing or shooting.

In some embodiments, the pharmaceutical composition is administered to treat trigeminal neuralgia (sometimes called tic douloureux). The main symptom of trigeminal neuralgia is a sudden, severe, stabbing, shooting pain on one side of the face. The pain manifests in intermittent episodes lasting form seconds to minutes. The attacks can occur several times a day for weeks or months followed by a period that is pain free.

In some embodiments, the pharmaceutical composition may include one or more Caᵥ3.3 inhibitors. For example, a Caᵥ3.3 targeting peptide may be used in conjunction with a Caᵥ3.3 targeting antibody and/or a Caᵥ3.3 targeting oligonucleotide.

In some embodiments, the Caᵥ3.3 inhibitor pharmaceutical composition may include an additional pain alleviating agent. In some embodiments, the additional pain alleviating agent may be administered at the same time as the Caᵥ3.3 inhibitor pharmaceutical composition. In some embodiments, the additional pain reliever agent may be administered in sequence with the Caᵥ3.3 inhibitor pharmaceutical composition. Additional pain reliever components may be an anticonvulsants, antispasmodics, or anticholinergics. Anticonvulsant drugs include, but are not limited to, carbamazepine, oxcarbazepine, topiramate, valproic acid, pregabalin, lamotrigine, phenytoin, clonazepam, and gabapentin. Examples of antispasmodic drugs include, but are not limited to, baclofen and lioresal. Examples of anti cholinergics include but are not limited to atropine, doxepin, flavoxate, tiotropium, and tricyclic antidepressants. Other additional pain reliever agents include, but are not limited to, opioids, botulinum toxin A injections, and glycerol injections.

In some embodiments, the Caᵥ3.3 inhibitor pharmaceutical composition may be administered in combination with other pain-relieving techniques. In some embodiments the Caᵥ3.3 inhibitor pharmaceutical composition may be administered in sequence with other pain-relieving techniques. Other pain-relieving techniques include, but are not limited to, microvascular decompression, brain stereotactic radiosurgery, balloon compression, and radiofrequency thermal lesioning.

In some embodiments, the Caᵥ3.3 inhibitor pharmaceutical composition may be more effective at providing pain relief for female subject than male subjects.

In some embodiments, pain relief can be characterized by a decrease in Caᵥ3.3 expression in a cell after treatment with a Caᵥ3.3 inhibitor or Caᵥ3.3 inhibitor pharmaceutical composition (FIGS. 3A and 3B). In some embodiments, the cell is from the trigeminal ganglia of the subject. Methods for qualitatively and quantitatively determining the Caᵥ3.3 expression are known in the art. Examples for determining Caᵥ3.3 expression include, but are not limited to, affinity pull down assays, tandem affinity purification assays, co-immunoprecipitation assays, and RNA-Seq assays. In some embodiments, trigeminal ganglia cells of from a female subject experience a greater decrease in Caᵥ3.3 than trigeminal ganglia cells from a male subject (FIGS. 3A and 3B).

In some embodiments, pain relief can be characterized by an increase in the mechanical withdrawal threshold of a subject after treatment with a Caᵥ3.3 inhibitor or Caᵥ3.3 inhibitor pharmaceutical composition (FIG. 1A, 1B, and FIG. 2). Methods for qualitatively and quantitatively determining the mechanical withdrawal threshold are known in the art. For example, von Frey fiber filaments may be used to probe the mechanical withdrawal threshold of a subject when applied to the skin of a subject. In some embodiments, female subjects treated with a Caᵥ3.3 inhibitor experience a greater increase in mechanical withdrawal than male subjects treated with a Caᵥ3.3 inhibitor (FIG. 2).

The amount of Caᵥ3.3 inhibitor administered can vary depending on various factors including, but not limited to, the specific Caᵥ3.3 inhibitor, the weight, physical condition, and/or age of the subject, and/or the route of administration. Thus, the absolute weight of Caᵥ3.3 inhibitor included in a given unit dosage form can vary widely, and depends upon factors such as the species, age, weight, and physical condition of the subject, and/or the method of administration. Accordingly, it is not practical to set forth generally the amount that constitutes an amount of Caᵥ3.3 inhibitor effective for all possible applications. Those of ordinary skill in the art, however, can readily determine the appropriate amount with due consideration of such factors.

In some embodiments, the method can include administering sufficient Caᵥ3.3 inhibitor to provide a dose of, for example, from about 100 ng/kg to about 1000 mg/kg to the subject, although in some embodiments the methods may be performed by administering Caᵥ3.3 inhibitor in a dose outside this range. Thus in some embodiments, the method can include administering sufficient Caᵥ3.3 inhibitor to provide a minimum dose of at least 100 ng/kg such as, for example, at least 500 ng/kg, at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, or at least 50 mg/kg. In some embodiments, the method can include administering sufficient Caᵥ3.3 inhibitor to provide a maximum dose of no more than 1000 mg/kg such as, for example, no more than 500 mg/kg, no more than 250 mg/kg, no more than 100 mg/kg, no more than 75 mg/kg, no more than 50 mg/kg, no more than 25 mg/kg, no more than 20 mg/kg, no more than 15 mg/kg, no more than 14 mg/kg, no more than 13 mg/kg, no more than 12 mg/kg, no more than 11 mg/kg, or no more than 10 mg/kg. As used herein, the term “no more than” a reference amount means that the Caᵥ3.3 inhibitor is not absent but is present in an amount up to the reference amount.

In some embodiments, the method can include administering sufficient Caᵥ3.3 inhibitor to provide a dose characterized by a range having endpoints defined by any a minimum dose identified above and any maximum dose identified above that is greater than the selected minimum dose. For example, in some embodiments, the method can include administering sufficient Caᵥ3.3 inhibitor to provide a dose of from 1 mg/kg to 50 mg/kg, from 4 mg/kg to 40 mg/kg, from 4 mg/kg to 10 mg/kg, from 10 mg/kg to 50 mg/kg, from 10 mg/kg to 25 mg/kg, etc.

In certain embodiments, the method can include administering sufficient Caᵥ3.3 inhibitor to provide a dose that is equal to any minimum dose or any maximum dose listed above. Thus, for example, the method can include administering sufficient Caᵥ3.3 inhibitor to provide a dose of 1 mg/kg, 4 mg/kg, 10 mg/kg, 15 mg/kg, 20 mg/kg, 25 mg/kg, 50 mg/kg, 100 mg/kg, 500 mg/kg, etc.

A single dose may be administered all at once, continuously for a prescribed period of time, or in multiple discrete administrations. When multiple administrations are used, the amount of each administration may be the same or different. For example, a dose of 10 mg/kg per day may be administered as a single administration of 10 mg/kg, continuously over 24 hours, as multiple equal administrations (e.g., two 0.5 mg/kg administrations), or multiple administrations in which at least two are unequal (e.g., a first administration of 0.75 mg/kg followed by a second administration of 0.25 mg/kg). When multiple administrations are used to deliver a single dose, the interval between administrations may be the same or different.

In some embodiments, the active agent may be administered, for example, from a single dose to multiple doses per week, although in some embodiments the method can involve a course of treatment that includes administering doses of the active agent at a frequency outside this range. When a course of treatment involves administering multiple within a certain period, the amount of each dose may be the same or different. For example, a course of treatment can include a loading dose initial dose, followed by a maintenance dose that is lower than the loading dose. Also, when multiple doses are used within a certain period, the interval between doses may be the same or be different.

In certain embodiments, Caᵥ3.3 inhibitor may be administered as needed to manage and/or alleviate pain. Thus, for example, the Caᵥ3.3 inhibitor may be administered up to six times per day. FIG. 1A and FIG. 1B show that a dose of 10 mg/kg appears to increase in effect until about four hours after administration, after which the effect of the Caᵥ3.3 inhibitor decreases. The period and extent of efficacy may be tuned by increasing the dose administered and/or employing a multiple dosing strategy.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, particular embodiments may be described in isolation for clarity. Thus, unless otherwise expressly specified that the features of a particular embodiment are incompatible with the features of another embodiment, the particular features, configurations, compositions, or characteristics may be combined in any suitable manner in one or more embodiments. Thus, features described in the context of one embodiment may be combined with features described in the context of a different embodiment except where the features are necessarily mutually exclusive.

The words “preferred,” “preferably,” and “especially” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

The present invention is illustrated by the following examples. It is to be understood that the particular examples, materials, amounts, and procedures are to be interpreted broadly in accordance with the scope and spirit of the invention as set forth herein.

### EXAMPLES

All animals were housed in a well-ventilated rodent housing room (maintained at 20° C.-22° C.) with a reversed 10/14-hour dark/light cycle so that testing could be performed in their active period. All animals were housed for one week before the experiments. All animals had access to food and water ad libitum throughout the duration of the experiment. Low soybean content normal chow diet was provided.

### FRICT-ION Model of Trigeminal Neuropathic Pain

All surgeries were completed in a sterile environment under a surgical microscope in mice anesthetized with isoflurane (2-5%). The FRICT-ION model was induced as previously described (Montera, M. A. and Westlund, K. N. (2020). Bio-protocol 10(8): e3591) in male and female BALBc mice (20 to 25 g; 8-10 weeks; Harlan Laboratories, Inc., Indianapolis, Ind.) in under 10 minutes per mouse. Mechanical allodynia developed within one-week post-surgery as evaluated using von Frey filaments.

### Behavioral Assays

Mechanical threshold of the whisker pad area was tested before and after surgery with a modified up/down method using a graded series of von Frey fiber filaments as described previously (Montera, M. A. and Westlund, K. N. (2020). Bio-protocol 10(8): e3591; Zhang et al. 2019. Mol Pain 15:1744806919884498; Ma et al., 2012. Mol Brain 5:44)

### RNA Extraction and RNAseq

Male and female mice (8-10 weeks old) were subject to the FRICT-ION model of trigeminal neuropathic pain. At 10 weeks post-injury, mice were euthanized by anesthetic overdose with pentobarbital (50 mg/kg) and both ipsilateral and contralateral whole trigeminal ganglia (TG) were removed. TG were washed immediately in PBS and stored at −80° C. to preserve RNA. RNA was isolated using RNeasy Mini Kits (Qiagen, Hilden, Germany). Yield and quality of RNA was determined using a NANODROP spectrophotometer (Thermo Fisher Scientific, inc., Waltham, Mass.). RNA was only used for further analysis if 260/280 ratio was ˜2.0. Whole trigeminal ganglia RNA samples from three FRICT-ION injured and three naïve mice were sent to Quick Biology, Inc. (Pasadena, Calif.) for RNAseq library preparation, sequencing, and gene expression analysis. The reads were first mapped to the latest UCSC transcript set using Bowtie2 version 2.1.0 (Langmead, B. and Salzberg, S. (2012). Nature Methods 9:357-359) and the gene expression level was estimated using RSEM v1.2.15. Differentially expressed genes were identified using the edgeR program (Robinson et al., Bioinformatics 26(1):136-140 (2010); McCarthy et al., Nucleic Acids Research 40(10):4288-4297 (2012)). Genes showing altered expression with p<0.05 were considered differentially expressed.

### Peptide Synthesis and Administration

TAT-based cell-penetrating peptides were synthesized by GenScript Biotech Corp. (Piscataway, N.J.). The Cav3.3 inhibiting peptide TAT-C3P (SEQ ID NO:1, molecular weight 4311 g/mol) was administered to male and female mice via intraperitoneal injection at a dose of 10 mg/kg and monitored every hour for up to five hours post-injection. A control peptide TAT-C3D (SEQ ID NO:2, molecular weight 4006 g/mol), which does not block Cav3.3 channels, was used in control experiments. For all experiments the peptides were dissolved in distilled water.

### Western Blot

Twelve male and female mice (8-10 weeks old, six mice per sex) were subject to the FRICT-ION model of trigeminal neuropathic pain. Three mice of each sex were administered TAT-C3P peptide (SEQ ID NO:1) and the remaining mice were administered TAT-C3D (SEQ ID NO:2) following the methods described above. Four hours post injection, mice were euthanized with pentobarbital (50 mg/kg) and both ipsilateral and contralateral trigeminal ganglia (TG) were removed. TG were washed immediately in PBS and stored at −80° C.

The TG for each of the four groups were pooled together (n=3) for protein extraction and homogenized using a pestle and 500 μL of 1×RIPA buffer (Thermo Fisher Scientific, Inc., Waltham, Mass.). Samples were put on a rocking shaker for two hours and then centrifuged, and supernatant removed to a new tube. Samples were assayed for total protein. (Bradford, Thermo Fisher Scientific, Inc., Waltham, Mass.). Samples were then prepared for electrophoresis by mixing with 2× sample buffer and boiling for five minutes at 100° C. for denaturation.

Proteins were loaded on a 12% Tris-Glycine polyacrylamide gradient gel (Bio-Rad Laboratories, Inc., Hercules, Calif.) and transferred to a PVDF membrane (MilliporeSigma, Burlington, Mass.). Membrane was blocked for an hour with 5% non-fat milk in TB ST buffer at room temperature and incubated at 4° C. with anti-Caᵥ3.3 antibody overnight (Alomone Labs Ltd., Jerusalem, Israel, Cat #ACC-009, diluted 1:200 in 2.5% milk in TB ST). The membrane was subsequently washed with TBST and then incubated with anti-rabbit secondary with HRP for one hour at room temperature (Abcam ab6721, diluted 1:1000 in TBST). The washing was repeated and then the blot developed with chemiluminescent substrate (Thermo Fisher Scientific, Inc., Waltham, Mass.). The blot was then imaged using an ODYSSEY FC imaging system (Li-Cor, Inc., Lincoln, Nebr.). Signal intensity was normalized to actin, which was used as a loading control (Abcam ab8227, 1:2000). Signal intensity was analyzed using ImageJ (Schneider et al., Nature Methods 9:671-675 (2012)) for comparisons.

### Statistical Analysis

Male and female mice were analyzed separately. Whisker pad mechanical thresholds were averaged for FRICT-ION-injured mice receiving TAT-C3P or TAT-C3D peptides. The behavioral data were expressed as mean±SEM using two-way analysis of variance (ANOVA) with post-hoc testing with Tukey's multiple comparisons over time. A p-value of <0.05 was considered significant. Normalized fold change between TAT-C3P-peptide treated and control TAT-C3D-peptide controls was compared with t-tests for the Western blots. Statistical analysis of the transcript cluster-level data comparisons was done via paired t-tests.

### Central Nervous System Penetration Assay

A His-tagging method was used to assess the brain-penetrance of peptide therapeutics (Westlund et al., 2021, Neurobiol Pain 10:100067). Briefly, a His-tagged TAT-C3P peptide with the sequence GRKKRRQRRRPQEESNKEAREDAELDAEIELEMAQGHHHHHH (SEQ ID NO: 3) was ordered from Genscript. Protein content was assessed with Western blots using standard methods. Briefly, the sample protein was denatured, followed by gel electrophoresis to separate proteins by molecular weight. An anti-His-tag monoclonal antibody (C-terminal A01857, GenScript) that binds to the His-tag conjugated to the TAT-C3P peptide or anti-β-actin antibody (ab8226, Abcam, Cambridge, United Kingdom) was applied to the electrophoresis membrane. After washing off the antibody, specific secondary antibodies were added which recognized and bound to the primary antibodies. The secondary antibody was visualized through immunofluorescence probe attached to the secondary antibody, allowing indirect detection, validation, and semi-quantitative assessment of the specific target protein.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

TAT-C3P
SEQ ID NO: 1
GRKKRRQRRR PQEESNKEAR EDAELDAEIE LEMAQG
TAT-C3D
SEQ ID NO: 2
GRKKRRQRRR PQAVSSPARS GEPLHALSPR GTARSP
His-tagged TAT-C3P
SEQ ID NO: 3
GRKKRRQRRR PQEESNKEAR EDAELDAEIE LEMAQGHHHH HH

What is claimed is:

1. A pharmaceutical composition comprising:
a Caᵥ3.3 inhibitor comprising an inhibitory polynucleotide; and
a pharmaceutically acceptable carrier.

2. The pharmaceutical composition of claim 1, wherein the inhibitory polynucleotide comprises an anti-sense oligonucleotide that hybridizes to at least a portion of Cacnali or an RNA transcribed from a portion of Cacnali.

3. A method for alleviating pain in a subject, the method comprising:
administering to the subject a Caᵥ3.3 inhibitor in an amount effective to alleviate pain.

4. The method of claim 3, wherein the pain is chronic pain.

5. The method of claim 4, wherein the chronic pain comprises trigeminal neuropathic pain.

6. The method of claim 3, wherein the Caᵥ3.3 inhibitor is more effective in a female subject than in a male subject.

7. The method of claim 3, wherein the Caᵥ3.3 inhibitor comprises a polypeptide.

8. The method of claim 7, wherein the peptide comprises the amino acid sequence of SEQ ID NO:1 or a structurally similar amino acid sequence.

9. The method of claim 7, wherein the peptide comprises an antibody or binding fragment thereof.

10. The method of claim 3, wherein the Caᵥ3.3 inhibitor comprises an inhibitory polynucleotide.

11. The method of claim 10, wherein the inhibitory polynucleotide comprises an anti-sense oligonucleotide that hybridizes to at least a portion of Cacnali or an RNA transcribed from a portion of Cacnali.

12. The method of claim 3, further comprising administering to the subject at least one additional pharmaceutical composition in an amount effective to alleviate pain.

13. A method of reducing Caᵥ3.3 expression in a cell, the method comprising:
contacting the cell with a Caᵥ3.3 inhibitor in an amount effective to reduce Caᵥ3.3 expression.

14. The method of claim 13, wherein reducing expression of Caᵥ3.3 comprises reducing Caᵥ3.3 in a lysate of whole trigeminal ganglion cells.

15. The method of claim 13, wherein the Caᵥ3.3 inhibitor is more effective in the cell isolated from a female than in the cell isolated from a male.

16. The method of claim 13, wherein the Caᵥ3.3 inhibitor comprises a polypeptide.

17. The method of claim 16, wherein the peptide comprises the amino acid sequence of SEQ ID NO:1 or a structurally similar amino acid sequence.

18. The method of claim 16, wherein the peptide comprises an antibody or binding fragment thereof.

19. The method of claim 13, wherein the Caᵥ3.3 inhibitor comprises an inhibitory polynucleotide.

20. The method of claim 19, wherein the inhibitory polynucleotide comprises an anti-sense oligonucleotide that hybridizes to at least a portion of Cacnali or an RNA transcribed from a portion of Cacnali.

21. A method for increasing mechanical withdrawal threshold in a subject, the method comprising:
administering to the subject a Caᵥ3.3 inhibitor in an amount effective to increase the mechanical withdrawal threshold in the subject.

22. The method of claim 21, wherein the Caᵥ3.3 inhibitor is more effective in a female subject than in a male subject.

23. The method of claim 21, wherein the Caᵥ3.3 inhibitor comprises a polypeptide.

24. The method of claim 23, wherein the peptide comprises the amino acid sequence of SEQ ID NO:1 or a structurally similar amino acid sequence.

25. The method of claim 23, wherein the peptide comprises an antibody or fragment thereof.

26. The method of claim 21, wherein the Caᵥ3.3 inhibitor comprises an inhibitory polynucleotide.

27. The method of claim 26, wherein the inhibitory polynucleotide comprises an anti-sense oligonucleotide that hybridizes to at least a portion of Cacnali or an RNA transcribed from a portion of Cacnali.
